(19) United States
(12) Patent Application Publication
(10) Pub. No.: US 2025/0292925 A1
(43) Pub. Date: Sep. 18, 2025

(54) RADIATION PROTECTIVE GARMENT

(71) Applicant: Taylor MAYBERRY, Brookline, MA (US)

(72) Inventor: Taylor MAYBERRY, Brookline, MA (US)

(21) Appl. No.: 19/033,105

(22) Filed: Jan. 21, 2025

Related U.S. Application Data
(60) Provisional application No. 63/623,518, filed on Jan. 22, 2024.

(51) Int. Cl. G21F 3/025; A61B 6/10

(52) U.S. Cl. CPC G21F 3/025; A61B 6/107

(57) ABSTRACT

The present disclosure provides a one-piece garment for protection of a wearer from radiation. The garment comprises an upper body section, a collar section and a front flap section to shield a region of thyroid, a region of torso and a region of gonad of the wearer.

### CROSS REFERENCE TO RELATED APPLICATION

This application claims the benefit of and priority to U.S. Provisional Application No. 63/623,518, filed Jan. 22, 2024, the contents of which are hereby incorporated by reference in their entirety for all purposes.

### RELATED FIELD

This field of the invention and its embodiments relate to a medical garment. More particularly, the present disclosure relates to a medical garment that shield a wearer from radiation exposure.

### BACKGROUND OF THE INVENTION

Radiation protective garments are commonly used to shield healthcare workers, as well as their patients, from radiation exposure during diagnostic imaging. These types of garments are often designed in a multiple-piece shield apron. As such, these garments are often uncomfortable, cumbersome and restrictive.

For example, U.S. Pat. No. 4,196,355 discloses a two-piece radiation shield vest and skirt for protecting a wearer's upper and lower body, respectively, from radiation. The vest and skirt, however, fails to protect a wearer's neck, thereby leaving the thyroid gland exposed to the harmful effects of ionizing radiation. Further, the vest and skirt are in two separate pieces and cumbersome to put on. Additionally, the vest and skirt are heavy and bulky and requires adjustability for distributing the weight and minimizing fatigue.

Therefore, there remains a need for a garment that is radiation resistant and that protects the health care worker's neck and lower body from unwanted radiation exposure. The radiation protective garment of the present disclosure fulfills these and other needs in the art.

### SUMMARY OF THE INVENTION

The present invention is an improvement in radiation shield garments designed for use by workers exposed to radiation. It comprises a one-piece garment to provide 3-in-1 coverage of the most anatomically vulnerable areas, while offering the wearer with ease of use and comfort.

According to the invention, there is provided a garment for protection of a wearer from radiation comprising:
an upper body section comprising a back portion, a first front portion, a second front portion and a neck opening, wherein the upper body section is configured to shield a region of a torso of the wearer from radiation;
a collar section being secured to the upper body section and upstanding from a neck opening, wherein the collar section is configured to shield a region of a thyroid of the wearer; and
a front flap section comprising a first front flap portion extending downwardly from the front first portion and a second front flap portion extending downwardly from the second front portion, wherein the front flap section is configured to shield a region of a gonad of the wearer;
wherein the garment is in one piece.

In some embodiments, the upper body section comprises at least one front pocket. In some embodiments, the upper body section comprises a holing element. In some embodiments, the body section comprises a first arm hole and a second arm hole.

In some embodiments, the upper body section comprises at least one first fastening means for releasably fastening a first vertical side edge of the first front portion and a second vertical side edge of the second front portion in a closed position. In some embodiments, the at least one first fastening means comprises a pair of complimentary assemblies comprising a first assembly integrally coupled to the first vertical side edge of the first front portion and a second assembly integrally coupled to the second vertical side edge of the second front portion. In some embodiments, the at least one first fastening means comprises at least one Velcro®, zipper or snap fastener.

In some embodiments, the collar section comprises a first distal end and a second distal end. In some embodiments, the collar section comprises at least one second fastening means comprising a pair of complimentary assemblies, wherein the pair of complimentary assemblies comprise a first assembly integrally coupled to the first distal end of the collar section and a second assembly integrally coupled to the second distal end of the collar section. In some embodiments, the at least one second fastening means comprises at least one Velcro®, zipper or snap fastener.

In some embodiments, the front flap section comprises at least one third fastening means for releasably fastening a first vertical side edge of the first front flap portion and a second vertical side edge of the second front flap portion in a closed position. In some embodiments, wherein the at least one third fastening means comprises a pair of complimentary assemblies comprising a first assembly integrally coupled to the first vertical side edge of the first front flap portion and a second assembly integrally coupled to the second vertical side edge of the second front flap portion. In some embodiments, the at least one third fastening means comprises at least one Velcro®, zipper or snap fastener.

In some embodiments, the first front portion of the upper body section and the first front flap portion of the front flap section form one continuous first panel, and the second front portion of the upper body section and the second front flap portion of the front flap section form one continuous second panel. In some embodiments, the first panel comprises at least one fastening means for releasably fastening a first vertical side edge of the first panel and a second vertical side edge of the second panel in a closed position. In some embodiments, the at least one fastening means comprises a pair of complimentary assemblies comprising a first assembly integrally coupled to the first vertical side edge of the first panel and a second assembly integrally coupled to the second vertical side edge of the second panel. In some embodiments, the at least one fastening means comprises at least one Velcro®, zipper or snap fastener.

In some embodiments, the main section is in a vest shape. In some embodiments, the garment is a garment for medical applications. In some embodiments, the garment allows the wearer to move freely in X, Y, Z spatial planes.

In some embodiments, the garment comprises a uni-layer or a multi-layer. In some embodiments, the multi-layer comprises at least one layer comprising radiation protective material. In some embodiments, the garment comprises an outer layer and an inner layer, wherein the outer layer comprises a flexible sterile fabric material and the inner layer comprises a radiation protective material.

In some embodiments, the garment comprises a radiation protective material. In some embodiments, the radiation protective material is operable for at least one of dissipating, absorbing, neutralizing, and deflecting radiation. In some embodiments, the collar section comprises at least one radiation protective material configured to protect a wearer's thyroid. In some embodiments, the upper body section comprises at least one radiation protective material configured to protect a wearer's chest. In some embodiments, the front flap section comprises at least one radiation protective material of the front flap section configured to protect a wearer's reproductive organs.

In some embodiments, the radiation protective material comprises an organic polymer. In some embodiments, the organic polymer comprises at least one of polyvinyl, polyolefin, polyester, polyacetate, and/or at least one of copolymers of polyvinyl, polyolefin and/or polyester, and/or at least one of polyacetate, polyvinyl chloride, polypropene and/or ethyl vinyl acetate. In some embodiments, the radiation protective material comprises a fibrous material comprising composite filaments. In some embodiments, the composite filaments comprises a composite material comprising a radiopaque substance. In some embodiments, the composite material comprises an inorganic material. In some embodiments, the inorganic material comprises one or several metals in oxidized form, elemental form, an alloy thereof, or salt form. In some embodiments, the composite material comprises an organic material. In some embodiments, the organic material comprises a thermoplastic polymer selected from polyvinyl, polyolefin, polyester, polyacetate, or copolymers thereof. In some embodiments, the radiopaque substance comprises one or more metals in oxidized form, elemental form, as an alloy, or in salt form in combination with an organic polymer. In some embodiments, the radiopaque substance is selected from the group consisting of actinium, antimony, barium, bismuth, bromine, cadmium, cerium, cesium, gold, iodine, indium, iridium, lanthanum, lead, mercury, molybdenum, osmium, platinum, pollonium, rhenium, rhodium, silver, strontium, tantalum, tellurium, thallium, thorium, tin, wolfram, and zirconium. In some embodiments, the amount of the radiopaque substance of the filaments is more than 25% by weight of the total weight of the filaments and less than 90% by weight of the filaments. In some embodiments, the garment comprises one or more layers of the radiation protective material.

### BRIEF DESCRIPTION OF THE FIGURES

The invention will be more clearly understood from the following description of some embodiments thereof, given by way of example only, with reference to the accompanying drawings, in which:

FIG. 1 is a front view of a radiation protective garment according to the invention;

FIG. 2 is a back view of a radiation protective garment shown in FIG. 1 in a closed configuration;

FIG. 3A is a front view of the radiation protective garment shown in FIG. 1 as it appears when worn by a wearer; FIG. 3B is a back view of the radiation protective garment shown in FIG. 1 as it appears when worn by a wearer; FIG. 3C is a side view of the radiation protective garment shown in FIG. 1 as it appears when worn by a wearer.

### DETAILED DESCRIPTION OF THE INVENTION

The following description focuses on embodiments of the present invention applicable for medical applications, such as protection for radiation from medical imaging such as X-ray radiography, fluoroscopy, angiography, computed tomography (CT), magnetic resonance imaging (MRI), nuclear medicine tomography (such as SPECT), and position emission tomography (PET). As will be apparent, the features and attributes of the specific embodiments disclosed herein may be combined in different ways to form additional embodiments, all of which fall within the scope of the present disclosure.

Throughout the present disclosure, the term “about” may be used in conjunction with numerical values and/or ranges. The term “about” is understood to encompass values near to a recited value and within an acceptable degree of error in the art. For example, “about 40 [units]” may mean within ±10%, ±9%, ±8%, ±7%, ±6%, ±5%, ±4%, ±3%, ±2%, ±1%, less than ±1%, or any other value or range of values therein or there below.

The present invention relates to a radiation protective garment. More specifically, the invention relates to a medical garment that incorporates a radiation resistant and/or dissipating material into select portions of the garment. The garment further includes an upstanding collar that protects the wearer's thyroid from radiation exposure. In one embodiment, the collar includes a Velcro® type fastener that prevents the collar from sagging. The garment is both sterile and disposable. The various components of the present invention, and the manner in which they interrelate, are described in greater detail hereinafter.

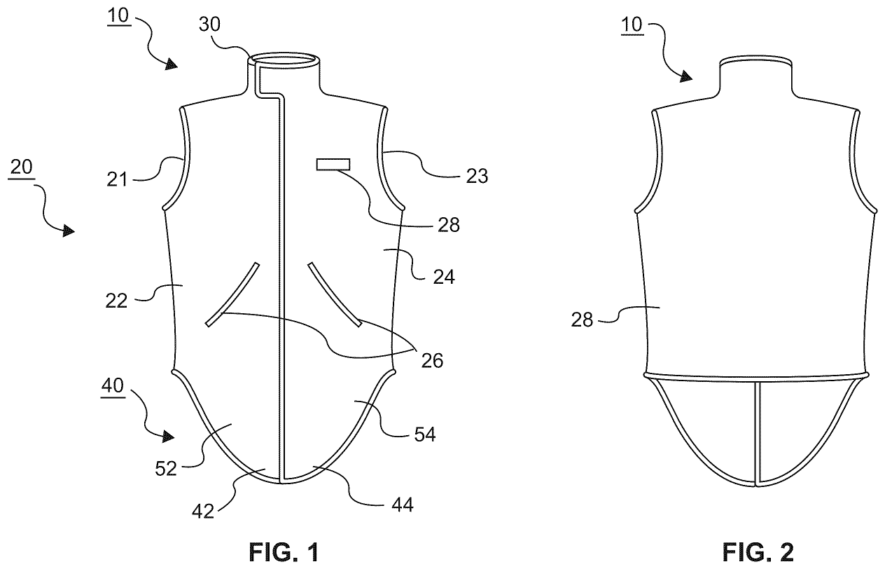

With reference to FIGS. 1 and 2, an embodiment of the radiation protective garment 10 of the present disclosure is depicted. The garment 10 includes an upper body section 20 comprising a back portion 28, a first front portion 22 and a second front portion 24, wherein the upper body section 20 is configured to shield a region of a torso of the wearer from radiation; a collar section 30 being secured to the upper extent of the upper body section 20 and upstanding from a neck opening, wherein the collar section 30 is configured to shield a region of a thyroid of the wearer; and a front flap section 40 comprising a first front flap portion 42 extending downwardly from the front first portion 22 and a second front flap portion 44 extending downwardly from the second front portion 24, wherein the front flap section 40 is configured to shield a region of a gonad of the wearer; wherein the garment is in one piece. In some embodiments, the upper body section 20 comprises at least one front pocket 26. In some embodiments, the upper body section 20 comprises a holding element 28 configured for holding or clipping a personal badge, e,g., an ID card or badge. In some embodiments, the holding element 28 may be in a form of a pocket, a strip, or any combination thereof. In some embodiments, the holding element 28 may be situated in the upper portion of the upper body section 20. In some embodiments, the upper body section 20 comprises a first arm hole 21 and a second arm hole 23.

In some embodiments, the upper body section 20 comprises at least one first fastening means for releasably fastening a first vertical side edge of the first front portion 22 and a second vertical side edge of the second front portion 24 in a closed position. In some embodiments, the at least one first fastening means comprises a pair of complimentary assemblies comprising a first assembly integrally coupled to the first vertical side edge of the first front portion 22 and a second assembly integrally coupled to the second vertical side edge of the second front portion 24. In some embodiments, the at least one first fastening means comprises at least one Velcro®, zipper or snap fastener.

In some embodiments, the collar section 30 comprises a first distal end and a second distal end. In some embodiments, the collar section comprises at least one second fastening means comprising a pair of complimentary assemblies, wherein the pair of complimentary assemblies comprise a first assembly integrally coupled to the first distal end and a second assembly integrally coupled to the second distal end. In some embodiments, the two ends of the collar section 30 are brought together such that the collar section fits securely about the wearer's neck. In some embodiments, the ends of the collar section 30 are then similarly secured via the Velcro® fasteners at the ends of collar section 30. This arrangement keeps the collar section 30 propped up during use and ensures that the lower neck region (including the thyroid) are adequately shielded from radiation.

In some embodiments, the front flap section 40 comprises at least one third fastening means for releasably fastening the first front flap portion 42 and the second front flap portion 44 in a closed position. In some embodiments, the at least one third fastening means comprises a pair of complimentary assemblies comprising a first assembly integrally coupled to a first vertical side edge of the first front flap portion and a second assembly integrally coupled to a second vertical side edge of the second front flap portion. In some embodiments, the at least one third fastening means comprises at least one snap fastener.

In some embodiments, the first front portion 22 of the upper body section 20 and the first front flap portion 42 of the front flap section 40 form one continuous first panel 52, and the second front portion 24 of the upper body section 20 and the second front flap portion 44 of the front flap section 40 form one continuous second panel 54. In some embodiments, the first panel 52 comprises at least one fastening means for releasably fastening a first vertical side edge of the first panel 52 and a second vertical side edge of the second panel 54 in a closed position. In some embodiments, the at least one fastening means comprises a pair of complimentary assemblies comprising a first assembly integrally coupled to the first vertical side edge of the first panel 52 and a second assembly integrally coupled to the second vertical side edge of the second panel 54. In some embodiments, the at least one fastening means comprises at least one Velcro®, zipper or snap fastener.

In some embodiments, the main section is in a vest shape. In some embodiments, the garment is a garment for medical applications.

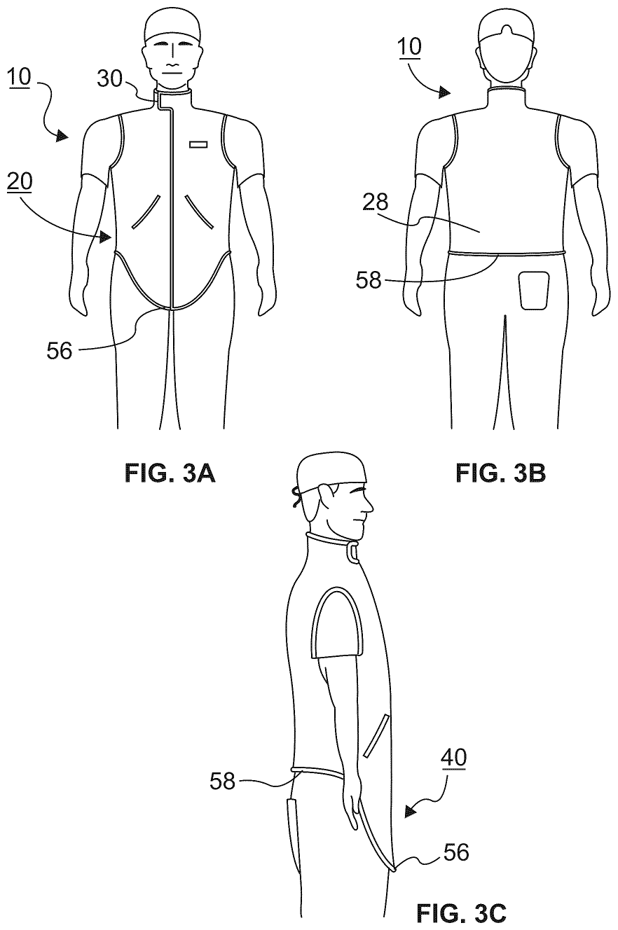

With reference to FIGS. 3A-3C, an embodiment of the radiation protective garment as it appears when worn by a wearer is depicted. With reference to FIG. 3A, the upper body section of the garment covers at least partially a torso region of a wearer and is configured to shield a region of a torso of the wearer from radiation; the collar section of the garment covers at least partially a neck region and is configured to shield a region of a thyroid of the wearer from radiation; and the front flap section cover at least partially the gonad region and is configured to shield a region of a gonad of the wearer from radiation. With reference to FIG. 3B, the back portion cover at least partially the back region of a wearer. In some embodiments, the bottom edge 58 of the back portion 28 is at the waist level of the wearer. With reference to FIG. 3C, the bottom edge 56 of the front flap section 40 is lower than the bottom edge of 58 of the back portion 27. The front flap section 40 is sufficiently long to cover the gonad region.

In some embodiments, the garment allows the wearer to move freely in X, Y, Z spatial planes. The garment allows the wearer to move freely in X, Y, and Z spatial planes, and the garment can be substantially weightless to the wearer.

In some embodiments, the garment of the present disclosure may be uni-layer or multi-layer with one layer comprising a radiation protective material. In some embodiments, the garment of the present disclosure comprises an outer layer and an inner layer, wherein the outer layer is formed from a flexible sterile fabric material and the inner layer is formed from a radiation protective material.

In some embodiments, the garment comprises one radiation protective material. In some embodiments, the garment comprises one or more radiation protective materials. In some embodiments, the upper body section, the collar section and the front flap section are formed from the same radiation protective material. In some embodiments, the upper body section and the collar section are formed from different radiation protective materials.

Any of a variety of radiation protective materials can be used in connection with the present invention.

In some embodiments, the radiation protective material comprises an organic polymer, such as a polypropylene material. The material described in U.S. Pat. No. 7,655,723, which is incorporated herein by reference. The material may either deflect, absorb, dissipate and/or neutralize the radiation as needed.

In some embodiments, the radiation protective material comprises a fibrous material with filaments including a radiopaque substance. The material described in U.S. Pat. No. 10,364,513, which is incorporated herein by reference. The filaments are structured in a regular pattern to form the radiation protective material. Such structure may be obtained by weaving or knitting. In some embodiments, the filaments may comprise a composite material including the radiopaque material. As such, it is relatively lightweight, depending on the quantity of radiopaque substance in the composite material. The composite material is lighter than lead based products of the same volume of material.

In some embodiments the composite material comprises an inorganic material, for example an inorganic composition, which includes one or several metals in oxidized form, elemental form, an alloy thereof, or salt form. In some embodiments, the composite material comprises an organic polymer matrix, such as a thermoplastic polymer. The organic polymer matrix may be selected from any kind of thermoplastic polymer, copolymers etc. In some embodiments, the thermoplastic polymer comprises polyvinyl, polyolefin, polyester, polyacetate, and/or copolymers thereof. In some embodiments, the thermoplastic polymer or copolymer comprises polyvinyl chloride, polypropene and/or ethyl vinyl acetate.

In some embodiments, the radiopaque substance is selected from the group consisting of actinium, antimony, barium, bismuth, bromine, cadmium, cerium, cesium, gold, iodine, indium, iridium, lanthanum, lead, mercury, molybdenum, osmium, platinum, pollonium, rhenium, rhodium, silver, strontium, tantalum, tellurium, thallium, thorium, tin, wolfram, and zirconium. Each element may be included in an amount of at least 2% by weight of the inorganic composition.

In some embodiments, the radiopaque substance comprises one or several different metals, in elemental form, in oxidized form, as an alloy, or in salt form, as the active radiopaque component. The metal in elemental form, in oxidized form, as an alloy, or in salt form may comprise at least one of: antimony, barium, bismuth, lanthanum, lead, tin, wolfram, and zirconium.

In some embodiments, the composite material comprises two metals, in elemental form, in oxidized form, as an alloy, or in salt form, that are selected within the groups of different embodiments.

In some embodiments, the radiation protective material comprises an inorganic material in combination with multiple polymers. In some embodiments, one polymer may. provide optimized properties to capsule the inorganic material, and another polymer may give the composite material optimized properties for the production technique, such as for weaving. Examples of such combinations include for example the polymer polyvinyl chloride, that provides for capsuling the inorganic material, and Bis(2-ethylhexyl) phthalate, which acts as plasticizer in the polymer matrix. Another example is where a multifilament yarn comprises monofilament fibers of polypropylene, incorporating the radiopaque components, in combination with a monofilament polyester fiber where the polyester fiber provides strength properties, such as enough strength for handling and/or manufacturing the yarn, e.g. through weaving. A combination of two or more of polymers is in some embodiments selected from the list of polymers above. In some embodiments, the composite material comprises an organic polymer matrix, such as listed above, in combination with at least one type of metal, in elemental form, in oxidized form, as an alloy, or in salt form. Thus, the composite material may be made of a mixture of the radiopaque material and the organic polymer matrix. As such, the radiopaque material may be embedded within the organic polymer matrix.

In some embodiments, the amount of the radiopaque substance of the composite material may be in the range of 15-90%, suitably in the range of 25-80%, and preferably more than 25% by weight of the total weight and less than 90% of the total weight of the composite material.

An example of a composite filament including a radiopaque substance is article number RONH 1030-785/2 from Roney Industri AB, Vellinge, Sweden, consisting of 61% of barium sulphate in a matrix of polyvinyl chloride and additives, having a diameter of 0.7 mm. Another example a composite filament including a radiopaque substance is Barilen 60 from Saxa Syntape GmbH, Luebnitz, Germany which is a multifilament yarn of 60% barium sulphate in a polypropylene matrix, supported by filaments of polyester.

In some embodiments, the structure of the radiation protective material, i.e. the structure of multiple individual filaments of the fibrous material relative to each other, is woven or knit. In some embodiments, at least one of the warp and the weft comprises filaments including the radiopaque substance, as described above. In some embodiments, filaments forming at least one of the warp and the weft comprise only filaments including the radiopaque substance, as described above, i.e. no other type of filaments. In still other embodiments, both the warp and the weft comprise filaments including the radiopaque substance, as described above, and optionally only such filaments and no other type of filaments. In those embodiments where only the warp or the weft comprise the radiopaque substance, the other filament may comprise a material such as cotton, polyester, nylon or a polyolefin, which does not include any radiopaque substance.

In some embodiment, a radiation protective material according to embodiments of the invention was made by utilizing a commercially available composite filaments including a radiopaque material (Barilen 60 from Saxa Syntape GmbH, Luebnitz, Germany which is a multifilament yarn of 60% barium sulphate in a polypropylene matrix, supported by filaments of polyester. There were 30 filaments at a fiber dimension of 2800-3200 m/kg where the single monofilament barium sulphate containing polypropene fiber had a diameter of about 0.06 mm). The filaments were structured into a regular pattern by weaving in twill in order to form the radiation protective material and achieve an air permeable textile material having as high radiation protection as possible.

In some embodiments, a garment for use in radiation protection comprises one or several layers of the radiation protective material.

Some embodiments of the invention provide for a comfortable radiation protective material that is lightweight and breathable. The material allows vapor, transport through the material, which significantly improves the comfort to the wearer. Furthermore, it is foldable without compromising the effectiveness of the radiation protection. Also, the material provides for easy-to-perform maintenance of any garment made thereof.

While the described invention has been described with reference to the specific embodiments thereof it should be understood by those skilled in the art that various changes may be made and equivalents may be substituted without departing from the true spirit and scope of the invention. In addition, many modifications may be made to adopt a particular situation, material, composition of matter, process, process step or steps, to the objective spirit and scope of the described invention. All such modifications are intended to be within the scope of the claims appended hereto.

Patents, patent applications, patent application publications, journal articles and protocols referenced herein are incorporated by reference in their entireties, for all purposes.

1. A garment for protection of a wearer from radiation, comprising:
an upper body section comprising a back portion, a first front portion, a second front portion and a neck opening, wherein the upper body section is configured to shield a region of a torso of the wearer from radiation;
a collar section being secured to the upper body section and upstanding from the neck opening, wherein the collar section is configured to shield a region of a thyroid of the wearer; and
a front flap section comprising a first front flap portion extending downwardly from the front first portion and a second front flap portion extending downwardly from the second front portion, wherein the front flap section is configured to shield a region of a gonad of the wearer;
wherein the garment is in one piece.

2. The garment of claim 1, wherein the upper body section comprises at least one front pocket; wherein the upper body section comprises a holing element; wherein the upper body section comprises a first arm hole and a second arm hole; wherein the upper body section comprises at least one first fastening means for releasably fastening a first vertical side edge of the first front portion and a second vertical side edge of the second front portion in a closed position; or any combination thereof.

3. (canceled)

4. (canceled)

5. (canceled)

6. The garment of claim 2, wherein the at least one first fastening means comprises a pair of complimentary assemblies comprising a first assembly integrally coupled to the first vertical side edge of the first front portion and a second assembly integrally coupled to the second vertical side edge of the second front portion.

7. (canceled)

8. The garment of claim 1, wherein the collar section comprises a first distal end and a second distal end; wherein the collar section comprises at least one second fastening means comprising a pair of complimentary assemblies, wherein the pair of complimentary assemblies comprise a first assembly integrally coupled to the first distal end of the collar section and a second assembly integrally coupled to the second distal end of the collar section; or any combination thereof.

9. (canceled)

10. (canceled)

11. The garment of claim 1, wherein the front flap section comprises at least one third fastening means for releasably fastening a first vertical side edge of the first front flap portion and a second vertical side edge of the second front flap portion in a closed position.

12. The garment of claim 11, wherein the at least one third fastening means comprises a pair of complimentary assemblies comprising a first assembly integrally coupled to the first vertical side edge of the first front flap portion and a second assembly integrally coupled to the second vertical side edge of the second front flap portion.

13. (canceled)

14. The garment of claim 1, wherein the first front portion of the upper body section and the first front flap portion of the front flap section form one continuous first panel, and the second front portion of the upper body section and the second front flap portion of the front flap section form one continuous second panel.

15. The garment of claim 14, wherein the first panel comprises at least one fastening means for releasably fastening a first vertical side edge of the first panel and a second vertical side edge of the second panel in a closed position.

16. The garment of claim 15, wherein the at least one fastening means comprises a pair of complimentary assemblies comprising a first assembly integrally coupled to the first vertical side edge of the first panel and a second assembly integrally coupled to the second vertical side edge of the second panel.

17. (canceled)

18. The garment of claim 1, wherein the upper body section is in a vest shape.

19. The garment of claim 1, wherein the garment is a garment for medical applications.

20. (canceled)

21. The garment of claim 1, wherein the garment comprises a uni-layer or a multi- layer.

22. The garment of claim 21, wherein the multi-layer comprises at least one layer comprising a radiation protective material.

23. The garment of claim 1, wherein the garment comprises an outer layer and an inner layer, wherein the outer layer comprises a flexible sterile fabric material and the inner layer comprises a radiation protective material.

24. The garment of claim 1, wherein the garment comprises a radiation protective material, wherein the radiation protective material is operable for at least one of dissipating, absorbing, neutralizing, and deflecting radiation.

25. (canceled)

26. The garment of any of the preceding claims, wherein the collar section comprises at least one radiation protective material configured to protect a wearer's thyroid; wherein the upper body section comprises at least one radiation protective material configured to protect a wearer's chest; wherein the front flap section comprises at least one radiation protective material of the front flap section configured to protect a wearer's reproductive organs.

27. (canceled)

28. (canceled)

29. The garment of any of the preceding claims, wherein the radiation protective material comprises an organic polymer comprising at least one of polyvinyl, polyolefin, polyester, polyacetate, and/or at least one of copolymers of polyvinyl, polyolefin and/or polyester, and/or at least one of polyacetate, polyvinyl chloride, polypropene and/or ethyl vinyl acetate; or wherein the radiation protective material comprises a fibrous material comprising composite filaments.

30. (canceled)

31. (canceled)

32. The garment of claim 31, wherein the composite filaments comprises a composite material comprising
a) a radiopaque substance comprising one or more metals in oxidized form, elemental form, as an alloy, or in salt form in combination with an organic polymer;
b) an inorganic material comprising one or several metals in oxidized form, elemental form, an alloy thereof, or salt form; or
c) an organic material comprising thermoplastic polymer selected from polyvinyl, polyolefin, polyester, polyacetate, or copolymers thereof.

33. (canceled)

34. (canceled)

35. (canceled)

36. (canceled)

37. (canceled)

38. The garment of claim 32, wherein the radiopaque substance is selected from the group consisting of actinium, antimony, barium, bismuth, bromine, cadmium, cerium, cesium, gold, iodine, indium, iridium, lanthanum, lead, mercury, molybdenum, osmium, platinum, pollonium, rhenium, rhodium, silver, strontium, tantalum, tellurium, thallium, thorium, tin, wolfram, and zirconium.

39. The garment of claim 32, wherein the amount of the radiopaque substance of the filaments is more than 25% by weight of the total weight of the filaments and less than 90% by weight of the filaments.

40. (canceled)
